(19) United States
(12) Patent Application Publication
(10) Pub. No.: US 2024/0044913 A1
(43) Pub. Date: Feb. 8, 2024

(54) METHOD FOR MEASUREMENT OF CELL FREE NUCLEOPROTEIN CHROMATIN FRAGMENTS

(71) Applicant: Belgian Volition SRL (BE/BE), Isnes (BE)

(72) Inventors: Guillaume ROMMELAERE, Isnes (BE); Mhammed BOUGOUSSA, Isnes (BE); Heather WILSON-ROBLES, Henderson, NV (US); Marielle HERZOG, Isnes (BE)

(21) Appl. No.: 18/253,768

(22) PCT Filed: Nov. 25, 2021
(86) PCT No.: PCT/EP2021/082939

§ 371 Date: May 19, 2023

Related U.S. Application Data
(60) Provisional application No. 63/118,329, filed on Nov. 25, 2020.

(51) Int. Cl. G01N 33/68; G01N 33/574; G01N 1/40

(52) U.S. Cl. CPC G01N 33/6875; G01N 33/57484; G01N 1/4077

(57) ABSTRACT

The invention relates to methods for detecting or measuring a cell free nucleoprotein chromatin fragment in a serum or plasma sample involving centrifugation prior to analysis.

### FIELD OF THE INVENTION

The invention relates to a method for detecting and measuring the presence of cell free chromatin fragments and the use of such measurements for the detection and diagnosis of disease.

### BACKGROUND OF THE INVENTION

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, 2007).

Normal cell turnover in the body involves the creation of new cells by cell division to replace cells that die. Cell death can occur by trauma as well as by various recognized mechanisms including apoptosis, necrosis, necroptosis, autophagy, ferroptosis, parthonatos, pyroptosis, NETosis and others. During cell death, chromatin is broken down into protein and nucleoprotein fragments. The most common nucleoprotein chromatin fragments are mononucleosomes and oligonucleosomes. Under normal conditions, chromatin fragments are metabolised and the level of circulating cell free nucleosomes found in healthy subjects is reported to be low. Elevated levels are found in subjects with a variety of conditions including many cancers, auto-immune diseases, inflammatory conditions, stroke and myocardial infarction (Holdenrieder & Stieber, 2009). Other cell free circulating protein and nucleoprotein chromatin fragments are reported including fragments comprising transcription factors or other non-histone chromatin protein fragments which may circulate free or bound to other chromatin components including DNA and/or nucleosomes (WO 2017/162755).

Mononucleosomes and oligonucleosomes can be detected by a number of methods. The most common method used is immunoassay, for example Enzyme-Linked ImmunoSorbant Assay (ELISA). Several ELISA methods have been reported (Salgame et al, 1997; Holdenrieder et al, 2001; van Nieuwenhuijze et al, 2003). The most commonly used nucleosome ELISA method is the Roche Cell Death assay which employs an anti-histone antibody as capture antibody and an anti-DNA antibody as detection antibody.

Nucleosome ELISA methods are used in cell culture, primarily as a method to detect apoptosis (Salgame et al, 1997; van Nieuwenhuijze et al, 2003) and are also commonly used for the measurement of circulating cell free nucleosomes in serum and plasma (Holdenrieder et al, 2001).

The epigenetic composition of circulating cell free nucleosomes in terms of their histone modification, histone variant (or isoform), DNA modification and adduct content have also been reported to be useful as blood based biomarkers in a wide variety of diseases including cancer, autoimmune diseases, inflammatory diseases, disorders associated with pregnancy and diseases associated with NETosis see WO 2005/019826, WO 2013/030577, WO 2013/030579, WO 2013/084002 and GB 2016403.4.

Circulating cell free protein chromatin fragments may also be measured. Examples of protein chromatin fragments known in the art include, without limitation, myeloperoxidase and neutrophil elastase. These protein chromatin fragments are useful measurements to detect circulating neutrophil extracellular traps or metabolites thereof.

We now report a method for the accurate determination of cell free nucleosomes and other nucleoprotein or protein chromatin fragments that leads to greatly improved biomarker performance.

### SUMMARY OF THE INVENTION

According to a first aspect of the invention there is provided a method for detecting or measuring a cell free nucleoprotein chromatin fragment in a serum or plasma sample obtained from a subject which comprises the steps of:
(i) centrifuging the serum or plasma sample; and
(ii) analysing the supernatant liquid for a cell free nucleoprotein chromatin fragment.

According to a further aspect of the invention there is provided a method for assessing or detecting a disease state of a subject which comprises the steps of:
(i) centrifuging a serum or plasma sample obtained from the subject;
(ii) analysing the supernatant liquid of the centrifuged serum or plasma sample for a nucleoprotein chromatin fragment; and
(iii) using the presence or the amount of nucleoprotein chromatin fragment as an indicator of the disease state of the subject.

### BRIEF DESCRIPTION OF THE FIGURES

FIG. 1: Plasma levels of nucleosomes containing histone isoform H3.1 measured by immunoassay with or without prior centrifugation of the plasma sample for 2 minutes at 14000×g (a) for 82 plasma samples taken from healthy subjects, and (b) for 74 plasma samples taken from subjects diagnosed with Non-Hodgkin's Lymphoma (NHL).

FIG. 2: The effect of prior centrifugation of plasma samples for 2 minutes at 14000×g on (a) the dot plot and (b) the ROC curve for the detection of subjects with NHL using levels of nucleosomes containing histone isoform H3.1 as a biomarker.

FIG. 3: Plasma levels of nucleosomes containing histone isoform H3.1 measured by immunoassay with or without prior centrifugation of plasma samples for 2 minutes at 14000×g for 32 plasma samples taken from subjects diagnosed with COVID-19. (a) Results expressed in concentration (ng/ml) and (b) results expressed in signal output of the immunoassay (relative light units or RLU).

FIG. 4: Plasma levels of nucleosomes containing histone modification H3R8-Cit measured by immunoassay with or without prior centrifugation of plasma samples for 2 minutes at 14000×g (a) for 19 plasma samples taken from healthy subjects, and (b) for 32 plasma samples taken from subjects diagnosed with COVID-19.

FIG. 5: Levels of nucleosomes containing histone isoform H3.1 measured in plasma and serum samples taken from 2 healthy dogs with and without prior centrifugation of the sample at 14000×g. (a) and (b) in fresh (never frozen) plasma and serum samples taken from Dog 1 and Dog 2 respectively; and (c) and (d) in the same samples following a freeze/thaw cycle.

### DETAILED DESCRIPTION OF THE INVENTION

Cell free nucleosomes and other chromatin fragments are released into the circulation on fragmentation of chromatin on cell death. Circulating cell free nucleosome levels are low in healthy subjects. Elevated levels are found in subjects with a variety of conditions involving cell death including many cancers, auto-immune diseases, inflammatory conditions, stroke and myocardial infarction (Holdenrieder & Stieber, 2009). Many infections, such as viral and bacterial infections, initiate cell death through a variety of mechanisms including cell binding and entry, endosomal TLR3 activation and gene expression, thereby increasing the number of circulating nucleosomes in the blood.

Another source of circulating cell free chromatin and nucleosomes arises from the production of neutrophil extracellular traps (NETs) by neutrophil cells as part of the immune response, for example in response to infection or inflammation by NETosis. NETosis is a regulated form of cell death in which neutrophils extrude chromatin together with antibacterial proteins into the extracellular space. The chromatin that makes up NETs comprises long fibres of DNA wound around consecutive nucleosomes in a “beads on a string” nucleoprotein structure. The constituent histones are toxic to pathogens and the NETs chromatin is also decorated with anti-pathogen enzymes including myeloperoxidase and neutrophil elastase. The extruded chromatin fibres act as a net and can capture and kill bacteria and other pathogens to prevent them from spreading. In addition to phagocytosis of pathogens and antibody production against pathogens, NETosis is therefore an important component of the immune response.

NETs and NETs metabolites therefore contribute to the total cell free nucleosomes measured in a sample. When NETs are elevated, they may be the dominant component in the mixture of circulating cell free nucleosomes and they may include large nucleoprotein chromatin fragments.

As well as the background level of circulating nucleosomes that are measurable in healthy subjects, there are multiple sources of cell free nucleosomes that may contribute to the total nucleosomes measured in a sample including, without limitation, nucleosomes arising from apoptosis, necrosis or other regulated cell death of cancer cells or other damaged or diseased cells as well as NETs extruded by neutrophil cells and metabolites produced by the digestion of NETs. This mixture of circulating cell free nucleosomes is heterogeneous with respect to chromatin fragment size and structure. Thus, circulating nucleosomes may include mononucleosomes as well as oligonucleosomes comprising a string of 2, 3, 4 etc nucleosomes and large nucleoprotein chromatin fragments comprising many nucleosomes. Similarly, the epigenetic structure of different types of circulating nucleosomes also vary, for example with respect to the proteins to which they are adducted and their post translationally modified histone content. For example, chromatin of NETs origin may include anti-pathogen enzymes and are reported to be contain high levels of citrullinated histones. Circulating nucleosomes of cancer cell origin may contain less linker DNA than nucleosomes derived from healthy cells (WO 2021/038010).

Circulating chromatin fragments therefore comprise a complex mixture of nucleosomes and other nucleoproteins with a multiplicity of origins and structures. This complexity provides a wealth of biomarker moieties which greatly increases the usefulness of circulating nucleosomes as a biomarker for any particular disease or condition and separating circulating nucleosomes of different structure or origins is therefore useful. However, this complexity also presents technical challenges in the measurement of different components of the mixture that can be used as biomarkers of disease. The present inventors have developed a method for improving the performance of clinical nucleosome measurements for nucleosome biomarkers generally and sub-types of nucleosomes containing particular epigenetic structures as biomarkers of disease.

In current practice, blood samples taken for nucleosome analysis are processed as blood samples taken in the normal way used by the laboratory concerned for other routine blood tests. The samples used by different workers in the field are therefore prepared using typical blood sample preparation procedures with local variations according to whatever the local procedure for collecting samples happens to be (for example in choice of blood collection tube, time of storage of whole blood prior to centrifugation and time and force of centrifugation used). No special sample preparation procedures are used. For serum samples, whole venous blood is typically collected from a subject into a regular glass or plastic serum collection tube. The tube is left to coagulate for at least 20 minutes (and often up to 24 hours) and then typically centrifuged at a relative centrifugal force (RCF) of 1500-3000 (or 1500-3000×g) for approximately 10-15 minutes. The clear serum is then removed from the pellet and transferred to a storage tube. The serum sample may be analysed immediately or stored refrigerated or frozen for later analysis. For plasma samples, whole venous blood is typically collected from a subject into a regular glass or plastic plasma collection tube. The tube is centrifuged at 1500-3000 RCF (or 1500-3000×g) for approximately 10-15 minutes. The clear plasma is then removed from the pellet and transferred to a storage tube. The plasma sample may be analysed immediately or stored refrigerated or frozen for later analysis.

Serum or plasma samples collected for the analysis of nucleosomes are not currently centrifuged before analysis. Similarly, blood samples collected for the analysis of nucleosomes are currently centrifuged at untypically high RCF for blood separation. The reasons for this include (i) it is not deemed necessary and methods known in the art do not mention the need for prior centrifugation of serum or plasma samples, (ii) the centrifuges used for blood sample collection are not typically capable of achieving high RCF and (iii) high RCF centrifugation would be considered dangerous for standard blood collection tubes, especially if glass blood collection tubes are used. Typical sample collection procedures for cancer samples are described by Holdenrieder et al, 2004.

Similarly, blood samples used for the analysis of circulating NETs or NETS metabolites are collected using standard collection protocols involving a single centrifugation step at approximately 1500-3000×g.

The inventors have found that a marked improvement in nucleoprotein chromatin fragment biomarker performance is produced by centrifugation of the serum or plasma sample prior to analysis for a nucleoprotein chromatin fragment. The method described herein thus involves a step of processing a serum or plasma sample by centrifugation prior to analysis. This differs from current practice, where whole blood is taken from a subject and centrifuged to produce a serum or plasma sample which is then directly analysed without further processing. Therefore, according to a first aspect of the invention there is provided a method for detecting or measuring a cell free nucleoprotein chromatin fragment in a serum or plasma sample obtained from a subject which comprises the steps of:
(i) centrifuging the serum or plasma sample; and
(ii) analysing the supernatant liquid for a cell free nucleoprotein chromatin fragment.

In a preferred embodiment the cell free nucleoprotein chromatin fragment comprises a nucleosome.

References to “nucleosome” may refer to “cell free nucleosome” when detected in body fluid samples. It will be appreciated that the term cell free nucleosome throughout this document is intended to include any cell free chromatin fragment that includes one or more nucleosomes. In one embodiment, the cell free nucleoprotein chromatin (or cell free nucleosome) is a part of, or derived from, a neutrophil extracellular trap (NET).

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or DNA.

Methods of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells.

In one embodiment, the nucleosome comprises an epigenetic feature. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of: a post-translational histone modification, a histone variant or isoform, a modified nucleotide and/or proteins bound to a nucleosome in a nucleosome-protein adduct.

The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. They can be classed into a number of families which are subdivided into individual types. The nucleotide sequences of a large number of histone isoforms are known and publicly available for example in the National Human Genome Research Institute NHGRI Histone Database (Marino-Ramirez et al. The Histone Database: an integrated resource for histones and histone fold-containing proteins. Database Vol. 2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, isoforms of histone H2 include H2A1, H2A2, mH2A1, mH2A2, H2AX and H2AZ. In another example, histone isoforms of H3 include H3.1, H3.2 and H3t. In one embodiment, the histone isoform is H3.1.

The structure of nucleosomes can vary by post translational modification (PTM) of histone proteins. PTM of histone proteins typically occurs predominantly on the tails of the core histones and common modifications include acetylation, methylation or ubiquitination of lysine residues as well as methylation or citrullination of arginine residues and phosphorylation of serine residues and many others. Many histone modifications are known in the art and the number is increasing as new modifications are identified (Zhao and Garcia (2015) Cold Spring Harb Perspect Biol, 7: a025064). Therefore, in one embodiment, the epigenetic feature of the cell free nucleosome may be a histone post translational modification (PTM). The histone PTM may be a histone PTM of a core nucleosome, e.g. H3, H2A, H2B or H4, in particular H3, H2A or H2B. In particular, the histone PTM is a histone H3 PTM. Examples of such PTMs are described in WO 2005/019826.

For example, the post translational modification may include acetylation, methylation, which may be mono-, di- or tri-methylation, phosphorylation, ribosylation, citrullination, ubiquitination, hydroxylation, glycosylation, nitrosylation, glutamination and/or isomerisation (see Ausio (2001) Biochem Cell Bio 79: 693). In one embodiment, the histone PTM is selected from citrullination or methylation. In a further embodiment, the histone PTM is H3 citrulline (H3cit) or H4 citrulline (H4cit). In a yet further embodiment, the histone PTM is H3R8cit.

In one embodiment, the epigenetic feature of the nucleosome comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues (or 5-hydroxymethylcytosine residues or other nucleotides or modified nucleotides) than other nucleosomes. In one embodiment, the DNA modification is selected from 5-methylcytosine or 5-hydroxymethylcytosine.

In one embodiment, the epigenetic feature of the nucleosome comprises one or more protein-nucleosome adducts or complexes. A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art.

In one embodiment, the protein adducted to the nucleosome is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate.

All of the circulating nucleosomes and nucleosome moieties, types or subgroups described herein may be useful in the present invention. Furthermore, it will be understood that more than one epigenetic feature of cell free nucleosomes may be detected in methods of the invention. Multiple epigenetic features may be used as a combined biomarker.

In one embodiment, the serum or plasma sample is centrifuged at a high relative centrifugal force. The term “relative centrifugal force” (RCF) is used herein to describe the force of acceleration applied to a sample in a centrifuge. RCF is measured in multiples of the standard acceleration due to gravity at the Earth's surface (g-force or ×g). Thus, RCF and g-force may be used interchangeably. The RCF generated by a centrifuge is determined by the speed at which the centrifuge revolves (for example expressed in revolutions per minute or RPM) and the radius (R for example expressed in mm) of the centrifuge arm that is spinning according to the equation: RCF or g-force=1.12×R×(RPM/1000)².

We have found that the usefulness of measured circulating nucleosomes as a biomarker for any particular disease or condition is greatly improved by subjecting the serum or plasma sample to be measured to a centrifugation step prior to analysis. In particular, a high g-force centrifugation of 14000×g (or 14000 RCF) for 2 minutes was used prior to nucleosome measurement.

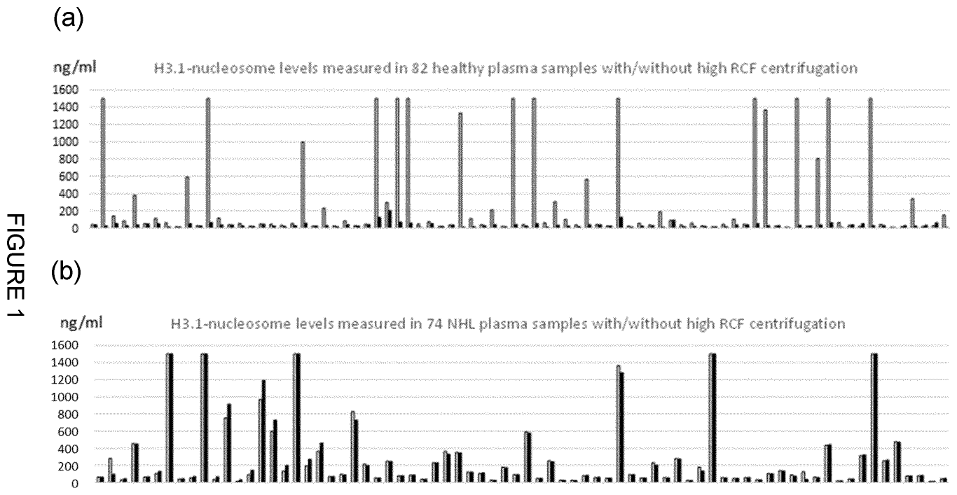

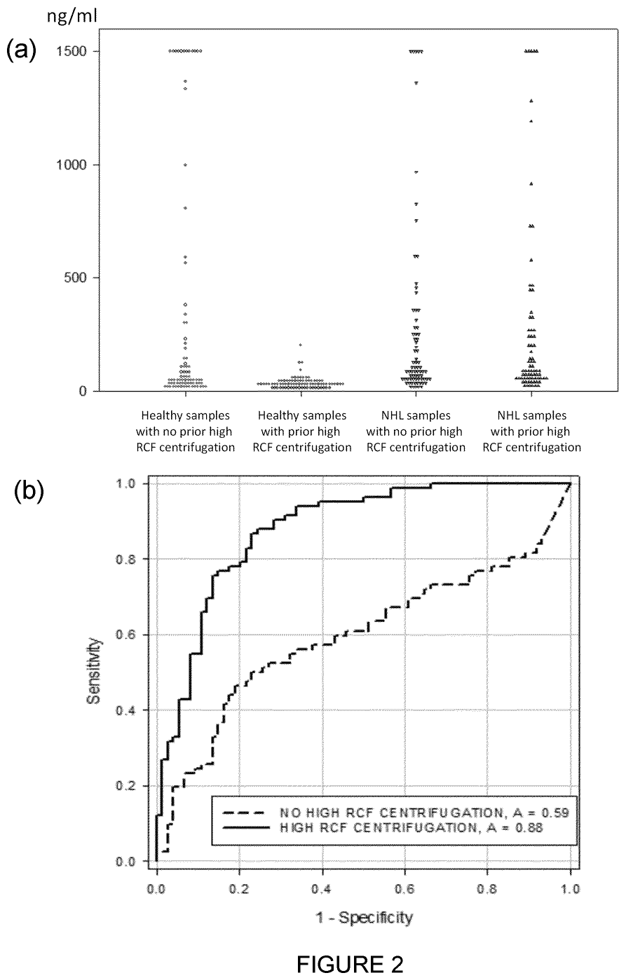

We measured the level of nucleosomes containing histone isoform H3.1 in plasma samples taken from 82 healthy subjects and from 74 subjects diagnosed with Non-Hodgkin's Lymphoma (NHL). We found that centrifugation of plasma samples prior to assay had a dramatic effect on the range of nucleosome levels measured in healthy subjects (a reduction of >90% for many samples) but little or no effect on the levels measured in subjects diagnosed with cancer. This dramatically narrowed the range of results observed for healthy subjects which greatly increased the difference in measured circulating cell free nucleosome levels between healthy and diseased subjects and greatly increased the Area Under the Curve (AUC) of receiver operating characteristic (ROC) curves (FIGS. 1 and 2). This finding that inclusion of a sample centrifugation step prior to sample analysis produced an improvement in biomarker performance by differentially reducing the level measured in healthy subjects was unexpected because sources of interference typically affect all samples or randomly affect some samples.

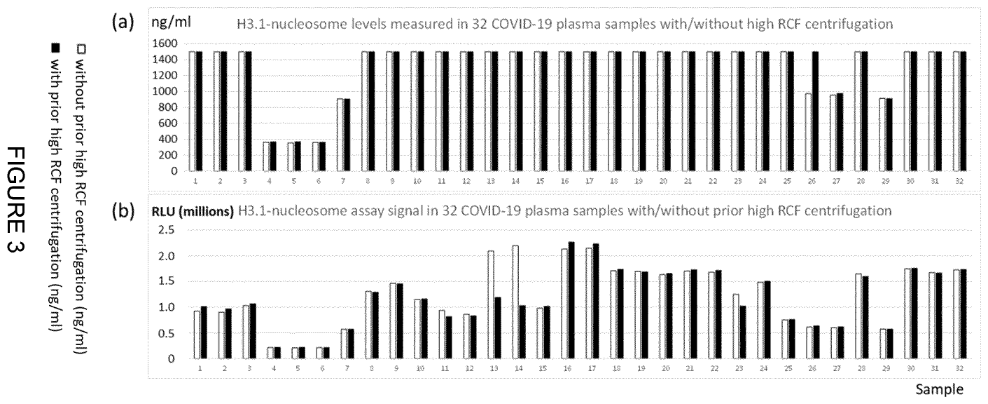

We have previously shown that patients suffering from COVID-19 have extremely high levels of circulating nucleosomes associated with excessive production of NETs. We therefore measured the level of nucleosomes containing histone isoform H3.1 in plasma samples taken from 32 subjects diagnosed with COVID-19 with and without prior high centrifugation. The levels found in most COVID-19 samples were above the highest standard used in the assay (1500 ng/ml). Of the measurable samples, none showed significant reduction in levels of nucleosomes containing histone isoform H3.1 after high g-force centrifugation (FIG. 3a). We therefore looked at the raw signal output produced by the assay itself in relative light units (RLU) and found that prior high g-force centrifugation resulted in a signal reduction of about half for 2 of the 32 samples tested (FIG. 3b).

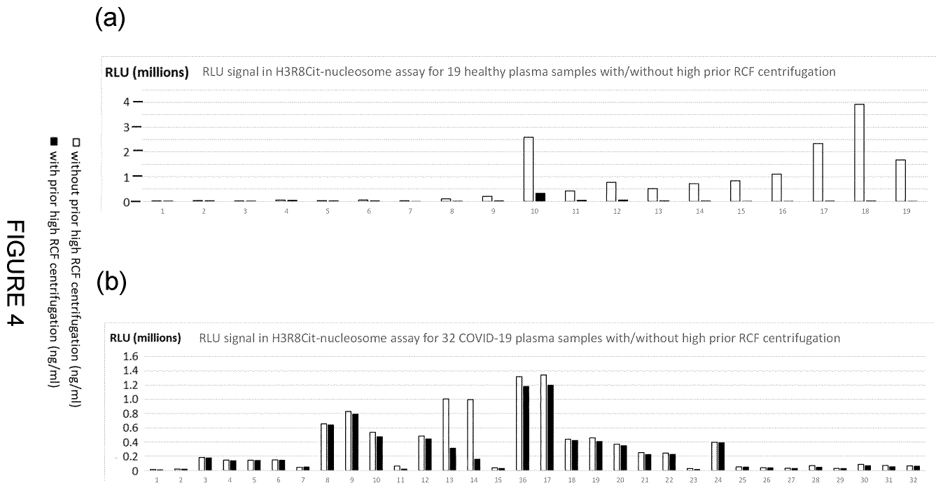

We then measured the level of nucleosomes containing the histone modification of citrullination of the arginine residue at position 8 of histone H3 (H3R8-Cit) in the samples taken from 32 subjects diagnosed with COVID-19 as well as in samples taken from 19 healthy subjects with and without prior high g-force centrifugation. The results showed that prior high g-force centrifugation resulted in a signal reduction of >50% in 14 of the 19 healthy samples, of >90% in 8 of the 19 healthy samples, and of >99% in 4 of the 19 healthy samples (FIG. 4a). In contrast, prior high g-force centrifugation resulted in a signal change of more than 50% for 3 of the 32 COVID-19 samples (samples 11, 13 and 14 in FIG. 4b).

As was observed for NHL, this effect resulted in dramatically improved ROC curves for the detection of COVID-19 in patients using either the level of circulating nucleosomes containing histone isoform H3.1 or H3R8-Cit as a biomarker.

The differential effect observed here for samples from healthy subjects compared to the lack of an observed effect on samples taken from subjects suffering from an excessive level of NETs due to the excessive NETosis associated with COVID-19, is even more surprising because NETs would be expected to comprise large nucleoprotein chromatin fragments that would be expected to be more subject to removal by centrifugation prior to analysis than the smaller nucleoprotein chromatin fragments present in healthy samples. Moreover, we have also observed a similar effect that nucleosome levels measured in samples taken from subjects suffering from severe sepsis (another disease associated with excessive NETosis) were extremely elevated and not attenuated by a prior centrifugation at 14000×g.

In one embodiment the sample is centrifuged at a relative centrifugal force of more than 1000×g, such as more than 2000×g. In preferred embodiments the sample is centrifuged at a high relative centrifugal force, such as above 5000×g, or above 6000×g, or above 7000×g, or above 8000×g, or above 9000×g, or above 10000×g, or above 11000×g, or above 12000×g, or above 13000×g, or above 14000×g. In a further embodiment, the sample is centrifuged at a relative centrifugal force of 5000-18000×g, such as 6000-15000×g, in particular, 10000-14000×g.

In one embodiment, the sample is centrifuged for at least 1 minute, such as between 1 and 10 minutes. In a further embodiment, the sample is centrifuged between 1 and 5 minutes, such as between 2 and 5 minutes. In a yet further embodiment, the sample is centrifuged for about 2 minutes.

In preferred embodiments, the sample is centrifuged for about 2 minutes at a relative centrifugal force of between 6000 and 15000×g.

As current practice involves the centrifugation of whole blood samples collected from subjects to produce serum or plasma, methods of the invention may involve two centrifugation steps including a first centrifugation of whole blood and a second centrifugation of the serum or plasma sample derived from the whole blood. Therefore, in one embodiment, the method additionally comprises: centrifuging a whole blood sample obtained from the subject to separate the blood cells from the serum or plasma and transferring the serum or plasma sample to a separate container prior to further centrifugation (i.e. prior to step (i) of the method).

According to a further aspect of the invention there is provided a method for detecting or measuring a circulating cell free nucleoprotein chromatin fragment in a serum or plasma sample taken from a subject which comprises the steps of:
(i) centrifuging a whole blood sample obtained from the subject to separate the blood cells from the liquid serum or plasma,
(ii) transferring the serum or plasma sample obtained in step (i) to a separate container,
(iii) centrifuging the serum or plasma sample in the separate container; and
(iv) analysing the supernatant liquid of the centrifuged serum or plasma sample obtained in step (iii) for a circulating cell free nucleoprotein chromatin fragment.

In one embodiment centrifugation of the serum or plasma sample is performed at a relative centrifugal force of more than 1000×g, such as more than 2000×g. In preferred embodiments the serum or plasma sample is centrifuged at a high relative centrifugal force, such as above 5000×g, or above 6000×g, or above 7000×g, or above 8000×g, or above 9000×g, or above 10000×g, or above 11000×g, or above 12000×g, or above 13000×g, or above 14000×g. In a further embodiment, the serum or plasma sample is centrifuged at a relative centrifugal force of 5000-18000×g, such as 6000-15000×g, in particular, 10000-14000×g.

In one embodiment, the serum or plasma sample is centrifuged for at least 1 minute, such as between 1 and 10 minutes. In a further embodiment, the serum or plasma sample is centrifuged between 1 and 5 minutes, such as between 2 and 5 minutes. In a yet further embodiment, the serum or plasma sample is centrifuged for about 2 minutes.

In one embodiment centrifugation of the whole blood sample is performed at a relative centrifugal force of more than 1500×g. In a further embodiment, the whole blood sample is centrifuged at a relative centrifugal force of 1500-3000×g.

In one embodiment, the whole blood sample is centrifuged for at least 1 minute, such as between 1 and 15 minutes. In a further embodiment, the whole blood sample is centrifuged between 5 and 15 minutes. In a yet further embodiment, the whole blood sample is centrifuged for about 10 minutes.

In a typical embodiment of the invention, serum or plasma is prepared by centrifugation of a whole blood sample for approximately 10 minutes using a g-force of 1500-3000×g. The centrifugation of the serum or plasma sample is then performed for approximately 2 minutes using a g-force of 6000-15000×g.

Methods of the invention involve centrifugation, therefore the container used for the sample must be suitable for use in a centrifuge. The container (such as a tube) is preferably constructed of a material which is both biocompatible and stable at high centrifugal forces. Suitable containers are known and available in the art, for example THERMO SCIENTIFIC NALGENE Oak Ridge High-Speed Polycarbonate Centrifuge Tubes. In one embodiment, the sample is retained in a container constructed of a polycarbonate material.

The serum or plasma sample may be frozen prior to centrifugation and analysis. This allows the sample to be stored. Therefore, in one embodiment, the serum or plasma sample is frozen and subsequently thawed prior to step (iii).

In an alternative embodiment, the serum or plasma sample used may be a fresh sample (i.e. not frozen). Results in Example 8 indicate that the artificial elevation of nucleosome levels is greater in frozen samples compared to fresh samples, therefore fresh samples may be used as an alternative sample type that may be used to provide an improvement in nucleoprotein chromatin fragment biomarker performance.

In a preferred embodiment the circulating cell free nucleoprotein chromatin fragment is a cell free nucleosome. Any method known in the art may be used for the analysis of the supernatant liquid for nucleosomes or other nucleoprotein chromatin fragments. In preferred embodiments the liquid is analysed for nucleosomes using an immunoassay. In a most preferred embodiment, the immunoassay is a double antibody or “sandwich” immunoassay employing two binders that bind to epitopes present on nucleosomes or other nucleoprotein chromatin fragments. In preferred embodiments the binders are directed to bind to an epitope present in a nucleoprotein chromatin fragment including antibodies or other binders directed to bind to nucleosomes, DNA, histone post translational modifications (PTMs), histone isoforms or other non-nucleosome chromatin components (for example, directed to bind to transcription factor) or any pairing of such antibodies or binders. Any specific binder may be used. In a preferred embodiment the binders are antibodies.

Diagnosis and Monitoring Methods

The level of circulating cell free nucleosomes and other circulating cell free nucleoprotein chromatin fragments can be useful for a variety of clinical purposes in a wide range of disease states. Both the level of circulating cell free nucleosomes and their epigenetic structure in terms of histone modification, histone variant, DNA modification and adduct composition are reported to be useful as blood based biomarkers in a wide variety of diseases including cancer, autoimmune diseases, inflammatory diseases, disorders associated with pregnancy and diseases associated with NETosis see WO 2005/019826, WO 2013/030577, WO 2013/030579, WO 2013/084002 and GB 2016403.4. Therefore, according to one aspect of the invention, there is provided a method for assessing or detecting a disease state of a subject which comprises performing a method for detecting or measuring a cell free nucleoprotein chromatin fragment as described herein.

According to a further aspect of the invention there is provided a method for assessing or detecting a disease state of a subject which comprises the steps of:
(i) centrifuging a serum or plasma sample obtained from the subject;
(ii) analysing the supernatant liquid of the centrifuged serum or plasma sample for a nucleoprotein chromatin fragment; and
(iii) using the presence or the amount of nucleoprotein chromatin fragment as an indicator of the disease state of the subject.

In one embodiment, the method additionally comprises: centrifuging a whole blood sample obtained from the subject to separate the blood cells from the serum or plasma and transferring the serum or plasma sample to a separate container prior to further centrifugation (i.e. prior to step (i) of the method).

In one embodiment of the invention, the method uses the presence or the amount of nucleoprotein chromatin fragment containing a particular epigenetic structure as an indicator of the disease state of the subject.

In a preferred embodiment, the cell free nucleoprotein chromatin fragment comprises a nucleosome. The nucleosome may contain an epigenetic structure. In one embodiment, the epigenetic structure is a histone modification, histone variant (or isoform), a DNA modification or a non-histone protein included in a nucleoprotein chromatin fragment.

Cell free nucleoprotein chromatin fragments measured or detected using methods of the invention may be used as a biomarker. The term “biomarker” means a distinctive biological or biologically derived indicator of a process, event, or condition. Biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

Methods of the invention can also be used to monitor the progression or progress of a disease in a subject to determine whether medical intervention is required. For example, if a subject is determined to have a mild stage of disease or is in remission, then the invention may be used for the purposes of monitoring disease progression for future development of a relapse. For example, if the method comprises a sample from a subject determined to have a mild stage of disease, then the biomarker level measurements can be repeated at another time point to establish if the biomarker level has changed.

In one embodiment, the disease state is selected from is selected from cancer, an infection, an autoimmune disease or an inflammatory disease. In a further embodiment, the disease state in cancer. Methods of the invention may be used with any cancer which include, for example, breast cancer, bladder cancer, colorectal cancer, skin cancer (such as melanoma), ovarian cancer, prostate cancer, lung cancer, pancreatic cancer, bowel cancer, liver cancer, endometrial cancer, lymphoma, oral cancer, head and neck cancer, leukaemia and osteosarcoma. In particular, the cancer may be a haematological cancer, such as leukaemia or lymphoma.

In an alternative embodiment, the disease state is an infection, such as a viral, bacterial, fungal or microbial infection. The Examples herein provide evidence that the method may also be used to detect infections, such as respiratory tract infections, for example COVID-19, or bacterial infections, such as sepsis.

The term “detecting” or “diagnosing” as used herein encompasses identification, confirmation, and/or characterisation of a disease state. Quantifying the amount of the biomarker present in a sample may include determining the concentration of the biomarker present in the sample. Methods of detecting, monitoring and of diagnosis according to the invention described herein are useful to confirm the existence of a disease, to monitor development of the disease by assessing onset and progression, or to assess amelioration or regression of the disease. Methods of detecting are also useful in methods for assessment of clinical screening, prognosis, choice of therapy, evaluation of therapeutic benefit, i.e. for drug screening and drug development.

The detection or measurement may comprise an immunoassay, immunochemical, mass spectroscopy, chromatographic, chromatin immunoprecipitation or biosensor method. In particular, detection and/or measurement may comprise a 2-site immunoassay method for nucleosome moieties. Such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. Also, detection and/or measurement may comprise a 2-site immunoassay, for example employing combinations of a labelled or immobilized: anti-nucleosome, anti-histone modification, anti-histone variant/isoform, anti-DNA modification or anti-adducted protein binding agent.

Detecting or measuring the level of the biomarker(s) may be performed using one or more reagents, such as a suitable binding agent. For example, the one or more binding agents may comprise a ligand or binder specific for the desired biomarker, e.g. nucleosomes or component part thereof, an epigenetic feature of a nucleosome, a structural/shape mimic of the nucleosome or component part thereof.

It will be clear to those skilled in the art that the terms “antibody”, “binder” or “ligand” as used herein are not limiting but are intended to include any binder capable of binding to particular molecules or entities and that any suitable binder can be used in methods of the invention. It will also be clear that the term “nucleosomes” is intended to include mononucleosomes, oligonucleosomes, NETs and any protein-DNA chromatin fragments that can be analysed in fluid media.

Methods of detecting biomarkers are known in the art. The reagents may comprise one or more ligands or binders, for example, naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

References to “subject” or “patient” are used interchangeably herein. The subject may be a human or an animal subject. In one embodiment, the subject is a human. In one embodiment, the subject is a (non-human) animal. The panels and methods described herein may be performed in vitro, in vivo or ex vivo.

Detecting and/or quantifying of the biomarker during analysis may be compared to a cut-off level. Cut-off values can be predetermined by analysing results from multiple patients and controls, and determining a suitable value for classifying a subject as with or without the disease. For example, for diseases where the level of biomarker is higher in patients suffering from the disease, then if the level detected is higher than the cut-off, the patient is indicated to suffer from the disease. Alternatively, for diseases where the level of biomarker is lower in patients suffering from the disease, then if the level detected is lower than the cut-off, the patient is indicated to suffer from the disease. The advantages of using simple cut-off values include the ease with which clinicians are able to understand the test and the elimination of any need for software or other aids in the interpretation of the test results. Cut-off levels can be determined using methods known in the art.

Detecting and/or quantifying of the biomarker during analysis may also be compared to a control. It will be clear to those skilled in the art that the control subjects may be selected on a variety of basis which may include, for example, subjects known to be free of the disease or may be subjects with a different disease (for example, for the investigation of differential diagnosis). The “control” may comprise a healthy subject.

Comparison with a control is well known in the field of diagnostics. The range of values found in the control group may be used as a normal or healthy or reference range against which the values found for test subjects can be compared. It will be understood that it is not necessary to measure controls levels for comparative purposes on every occasion. For example, for healthy/non-diseased controls, once the ‘normal range’ is established it can be used as a benchmark for all subsequent tests. A normal range can be established by obtaining samples from multiple control subjects without the disease and testing for the level of biomarker. Results (i.e. biomarker levels) for subjects suspected to have the disease can then be examined to see if they fall within, or outside of, the respective normal range. Use of a ‘normal range’ is standard practice for the detection of disease.

Diagnostic or monitoring kits are provided for performing methods of the invention. Such kits will suitably comprise one or more ligands for detection and/or quantification of the biomarker according to the invention, optionally together with instructions for use of the kit. In one embodiment, the kit comprises one or more containers that are suitable for use at high relative centrifugal forces.

According to a further aspect of the invention, there is provided a kit comprising one or more ligands for detection and/or quantification of cell free nucleoprotein chromatin fragments in a serum or plasma sample and one or more containers that are suitable for use at high relative centrifugal forces.

According to a further aspect of the invention, there is provided the use of a kit for detecting and/or quantifying of cell free nucleoprotein chromatin fragments comprising one or more ligands for detection and/or quantification of cell free nucleoprotein chromatin fragments and one or more containers that are suitable for use at high relative centrifugal forces. In a further embodiment, the cell free nucleoprotein chromatin fragments are detected in a serum or plasma sample.

It will be understood that the embodiments described herein may be applied to all aspects of the invention, i.e. the embodiment described for the methods may equally apply to the claimed kits and so forth.

The invention will now be illustrated with reference to the following non-limiting examples.

### Example 1

EDTA plasma samples were collected from 82 healthy subjects and from 74 subjects diagnosed with NHL by a biobank according to a standardized protocol. Whole blood samples were collected in standard EDTA plasma blood collection tubes. Each blood collection tube was centrifuged at 1500×g for 15 minutes within 2 hours of venipuncture. The liquid plasma supernatant was transferred to a cryovial and frozen at −80° C. until thawed for analysis.

In order to determine the effect of a prior centrifugation on observed nucleosome level results, each frozen plasma sample was thawed and (i) assayed for nucleosomes containing histone variant H3.1 without further processing and (ii) centrifuged at 14000×g for 2 minutes and the supernatant was assayed for nucleosomes containing histone variant H3.1.

Assay measurements for nucleosomes containing histone variant H3.1 were performed by immunoassay using an automated immunoassay instrument. Briefly, calibrant or sample (50 μl) was incubated with an acridinium ester labelled anti-nucleosome antibody (50 μl) and assay buffer (100 μl) for 1800 seconds at 37° C. Magnetic beads coated with an anti-histone H3.1 antibody (20 μl) were added and the mixture was incubated a further 900 seconds. The magnetic beads were then isolated, washed 3 times and magnetic bound acridinium ester was determined by luminescence output over 7000 milliseconds.

We found that prior centrifugation of samples had a dramatic effect on the range of nucleosome levels measured in healthy subjects (the normal range) but little or no effect on the levels measured in subjects diagnosed with cancer.

Centrifugation for 2 minutes at 14000×g prior to measurement of nucleosomes containing histone isoform H3.1 in samples taken from 82 healthy samples resulted in a reduction in observed results by more than 50% in 41 of the 82 subjects tested and by more than 90% in 24 of the 82 subjects tested (FIG. 1a). In contrast, the level of nucleosomes containing histone isoform H3.1 measured in samples taken from 74 subjects diagnosed with NHL was relatively unaffected with levels reduced by more than 50% in only 2 of the 74 samples (samples 2 and 62 in FIG. 1b). As shown in FIG. 2a, this effect dramatically increased the difference in measured circulating cell free nucleosome levels between healthy and diseased subjects and dramatically increased the Area Under the Curve (AUC) of receiver operating characteristic (ROC) curves (FIG. 2b). The clinical sensitivity for detection of NHL was, for example, increased by more than 30% at a specificity of 90%. The observed differential effect on the measured nucleosome levels in samples taken from healthy subjects compared to the much smaller effect on samples taken from cancer patients was unexpected as removal of a background interfering factor would be expected to occur for all samples or randomly. For example, any contamination that may occur due to poor sampling technique would be expected to occur randomly.

### Example 2

Plasma samples were taken from 32 subjects diagnosed with COVID-19 and stored frozen at −80° C. The samples were thawed for analysis and assayed for nucleosomes containing histone variant H3.1 by immunoassay with and without prior centrifugation for 2 minutes at 14000×g as described in EXAMPLE 1. The levels found in most samples were above the highest standard used in the assay (1500 ng/ml). Of the samples found to have levels <1500 ng/ml, none showed significant reduction in levels of nucleosomes containing histone isoform H3.1 after high g-force centrifugation (FIG. 3a). We therefore also looked at the raw signal output produced by the assay itself in relative light units (RLU) and found that prior high g-force centrifugation resulted in a signal reduction of about half for 2 of the 32 samples tested (samples 13 and 14 in FIG. 3b).

### Example 3

To investigate whether the observed effect was specific to measurements of nucleosomes containing histone isoform H3.1, we also measured the level of nucleosomes containing a different epigenetic signal. We selected nucleosomes containing citrullinated arginine at residue 8 of histone H3 (H3R8Cit) as another measurement for COVID-19 samples. We measured nucleosomes containing H3R8Cit by immunoassay using an immobilized anti-H3R8Cit antibody in combination with a labelled anti-nucleosome antibody, in the 32 samples taken from subjects diagnosed with COVID-19 as well as 19 samples taken from healthy subjects with and without prior high centrifugation for 2 minutes at 14000×g. All the samples were stored frozen at −80° C. and thawed for analysis.

The results showed that prior high g-force centrifugation resulted in a signal reduction of >50% in 14 of the 19 healthy samples, of >90% in 8 of the 19 healthy samples, and of >99% in 4 of the 19 healthy samples (FIG. 4a). In contrast, prior high g-force centrifugation resulted in a signal change of more than 50% for 3 of the 32 COVID-19 samples (samples 11, 13 and 14 in FIG. 4b).

### Example 4

We further investigated the effect of sample centrifugation on nucleosome results by conducting experiments using plasma samples taken from healthy subjects that were positive (i.e. found to contain high levels of plasma nucleosomes) by immunoassay when assayed without a prior high g-force centrifugation, but negative (i.e. had a much lower nucleosome level) following a prior high g-force centrifugation. The samples were stored frozen at −80° C. and thawed for analysis.

In the first experiment we centrifuged a 1 ml healthy sample at 14000×g for 2 minutes and measured plasma nucleosomes containing histone isoform H3.1 in 50 μl removed carefully from the top 250 μl of the supernatant, in 50 μl removed from the second 250 μl, in 50 μl removed from the third 250 μl and in 50 μl removed carefully from the bottom 250 μl of supernatant in the tube (which included the centrifugation pellet). The measured plasma nucleosome level with no prior centrifugation was >1500 ng/ml. The first, second and third supernatant fractions of the centrifuged plasma sample all contained less than 40 ng/ml nucleosomes. The bottom fraction including the pellet was found to contain 1293 ng/ml. Thus, nucleosome measurements made in the bottom 250 μl were subject to interference from the centrifugation pellet. We conclude that the interfering moieties present in plasma samples that were removed by centrifugation, were present in the pellet. We also conclude that pelleted material may unintentionally be included in a supernatant sample if the pellet is disturbed during collection.

### Example 5

We thawed a frozen healthy plasma sample, centrifuged the sample at 14000×g for 2 minutes and transferred a portion of the supernatant to another tube. We measured plasma nucleosomes containing histone isoform H3.1 by immunoassay immediately and on the following day following (another) freeze/thaw cycle. The level with no prior centrifugation was >1500 ng/ml. The level measured in the supernatant immediately following centrifugation was 40 ng/ml. The level measured the following day in the supernatant after a freeze/thaw cycle was 32 ng/ml. Thus, once prepared, the supernatant may be stored frozen before analysis. Therefore, in one embodiment of the invention a body fluid sample processed by a method of the invention may be stored until analyzed.

### Example 6

We thawed a frozen healthy plasma sample, centrifuged the sample at 14000×g for 2 minutes and measured nucleosomes containing histone isoform H3.1 in the supernatant by immunoassay. We then mixed the sample prior to re-analysis by immunoassay. The plasma nucleosome level with no prior centrifugation was >1500 ng/ml. The level measured in the supernatant following centrifugation was 40 ng/ml. The level measured following mixing of the centrifuged sample was >1500 ng/ml. From this result, together with the results described in EXAMPLE 4, we conclude that it is preferential to remove the supernatant from the pellet prior to storage as described in the EXAMPLE 5 above.

### Example 7

Two healthy samples whose measured levels of nucleosomes containing histone isoform H3.1 without prior centrifugation were >1500 ng/ml and 438 ng/ml, were thawed and centrifuged for 2 minutes at 2000×g, 3000×g, 5000×g, 7500×g or 14000×g. The nucleosome levels measured following centrifugation decreased markedly at all g-forces applied for both samples (Table 1). Thus, centrifugation of a serum or plasma sample prior to analysis for chromatin fragments is effective even using low g-forces.

We also observed that, nucleosome levels measured following centrifugation of the samples decreased further with increasing g-force of centrifugation from 63 to 34 and from 47 to 30 ng/ml. The mean level of nucleosomes containing histone isoform H3.1 determined for 82 healthy subjects in EXAMPLE 1 was 35 ng/ml. The higher results produced by centrifugation at g-forces below 5000×g are therefore significant in relation to healthy levels and will affect diagnostic performance. The variation between results produced by centrifugation at 7500 or 14000×g appeared small. We conclude that the method of the invention was effective for centrifugation at all g-forces applied and that optimal results were obtained by use of centrifugation forces of at least 5000×g.

TABLE 1
Nucleosome levels (ng/ml) following centrifugation
of plasma samples at various g-forces

| Centrifugation | | |
|---|---|---|
| force applied | Nucleosome level Sample 1 | Nucleosome level Sample 2 |
| No prior | >1500 | 438 |
| centrifugation | | |
| 2000 × g | 63 | 47 |
| 3000 × g | 55 | 46 |
| 5000 × g | 44 | 35 |
| 7500 × g | 34 | 31 |
| 14000 × g | 33 | 30 |

### Example 8

The experiments described in EXAMPLES 1-7 involved the use of frozen plasma samples. We therefore investigated the effect of freezing on the measured nucleosome level in EDTA plasma samples. We collected EDTA plasma samples from 14 healthy human volunteers and split each plasma sample into three aliquots. One aliquot was refrigerated, one aliquot was frozen at −20° C. and one aliquot was frozen at −80° C. We then measured the level of nucleosomes containing histone variant H3.1 in all 3 aliquots of each plasma sample. We found that freezing resulted in an increase in measured nucleosome levels for every sample with a mean increase of 53% and an increase of more than double for some samples (up to 144%).

Compared to the effect shown in FIGS. 1, 2 and 4, the effect described here, in EXAMPLE 8, is a smaller effect that affects all or most samples. Although smaller, the effect nonetheless results in a lower normal range for nucleosomes containing histone variant H3.1 for fresh plasma samples in comparison to frozen plasma samples and hence results in an improved AUC for the detection of disease. Therefore, if possible to use, fresh plasma is a preferable sample matrix for use in nucleosome tests compared to frozen plasma.

### Example 9

To investigate whether fresh (never frozen) serum and plasma samples are additionally subject to the same effect, we took blood samples from two healthy dogs into serum and plasma blood collection tubes. The whole blood samples were centrifuged to produce serum and plasma samples using the veterinarian's usual blood processing method. The fresh serum and plasma samples were assayed in duplicate for nucleosomes containing histone variant H3.1, (i) without further processing and (ii) the serum and plasma samples were centrifuged at 14000×g for 2 minutes and the supernatant was assayed for nucleosomes containing histone variant H3.1.

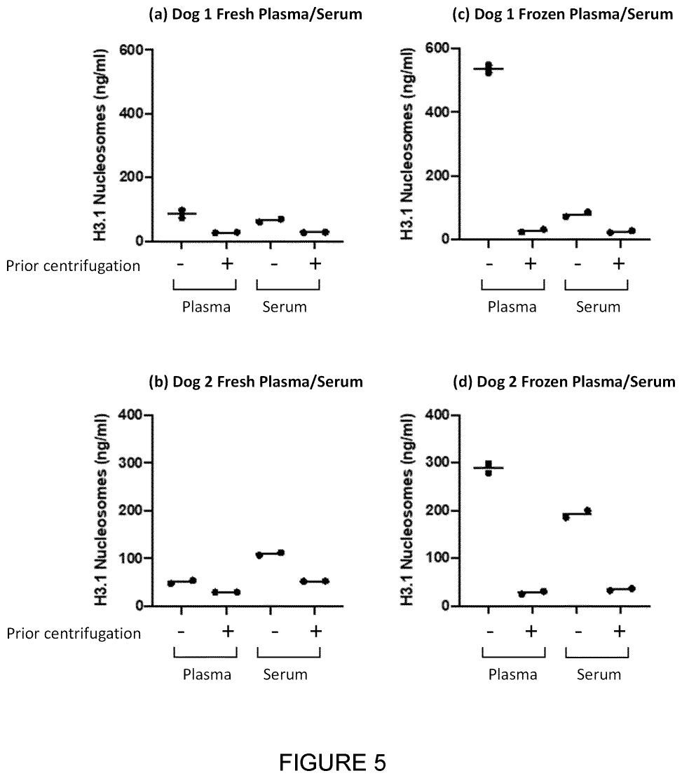

The results (shown in FIGS. 5a and 5b for Dog 1 and Dog 2 respectively) show that the level of measured nucleosomes containing histone variant H3.1 measured in fresh plasma or serum prepared from both dogs were significantly decreased following a prior centrifugation at 14000×g. The effect of the centrifugation step prior to serum or plasma analysis is therefore a feature of serum and plasma samples generally and is not restricted to frozen samples. The finding of artifactually elevated nucleosome levels in 2 of 2 healthy dogs tested is consistent with the finding of artifactually elevated nucleosome levels in healthy human subjects. The results also show that the effect occurs for multiple species and is not restricted to human samples.

### Example 10

The fresh plasma and serum samples prepared in EXAMPLE 9 from 2 healthy dogs were frozen and then assayed again (i) without further processing and (ii) the serum and plasma samples were centrifuged at 14000×g for 2 minutes and the supernatant was assayed for nucleosomes containing histone variant H3.1.

The results (shown in FIGS. 5c and 5d for Dog 1 and Dog 2 respectively) show that the measured level of nucleosomes containing histone variant H3.1 observed in both plasma and serum samples prepared from both dogs was significantly increased following a freeze/thaw cycle. The plasma samples were particularly susceptible to this effect and a freeze/thaw cycle of the plasma sample collected from both dogs resulted in an increase in the observed levels of nucleosomes of more than 500%. This finding is consistent with the high healthy nucleosome levels observed in frozen human plasma samples when assayed without a pre-spin step described in EXAMPLE 1 and EXAMPLE 3.

When the dog serum or plasma samples were centrifuged at 14000×g for 2 minutes and the supernatant was assayed for nucleosomes containing histone variant H3.1, the measured levels observed decreased to the same levels observed for the fresh (never frozen) samples when assayed following a prior pre-spin step in EXAMPLE 7.

We conclude, (i) both fresh and frozen plasma or serum samples may yield artifactually elevated results for chromatin fragment levels when measured without a prior centrifugation step, (ii) that addition of a prior centrifugation step removes the interference and reduces the measured level, (iii) that (a first) freezing and thawing of fresh serum or plasma samples leads to an artifactual increase in the measured levels of nucleosomes in the sample and that the size of the increase may be much larger than the size of the original nucleosome level measured and (iv) that addition of a prior centrifugation step removes the interference caused by freezing and thawing and reduces the measured level to the same level observed in fresh samples using an assay following a prior centrifugation step according to the method of the invention.

In conclusion, the potential clinical biomarker applications of circulating serum and plasma cell free nucleosome measurements have been known for several years (Holdenrieder et al, 2001). Despite this, nucleosome measurements have, so far, not been used for clinical purposes. The current inventors have shown for the first time that cell free nucleosome measurements, as currently performed, are subject to artifactual interference and that this interference can be removed by addition of a centrifugation step prior to the assay of the chromatin fragments. Moreover, the inventors have shown that removal of the interference using methods of the invention results in improved biomarker performance.

### REFERENCES

Herranz and Esteller, DNA methylation and histone modifications in patients with cancer: potential prognostic and therapeutic targets. Methods Mol Biol. 361:25-62, 2007
Holdenrieder et al, Nucleosomes in serum of patients with benign and malignant diseases. Int. J. Cancer (Pred. Oncol.): 95, 114-120, 2001
Holdenrieder et al, Circulating nucleosomes predict the response to chemotherapy in patients with advanced Non-Small Cell Lung Cancer. Clinical Cancer Research; 10, 5981-5987, 2004
Holdenrieder and Stieber, Clinical use of circulating nucleosomes. Critical Reviews in Clinical Laboratory Sciences; 46(1): 1-24, 2009
Salgame et al, An ELISA for detection of apoptosis. Nucleic Acids Research, 25(3), 680-681, 1997
van Nieuwenhuijze et al, Time between onset of apoptosis and release of nucleosomes from apoptotic cells: putative implications for systemic lupus erythematosus. Ann Rheum Dis; 62: 10-14, 2003

1. A method for detecting or measuring a cell free nucleoprotein chromatin fragment in a serum or plasma sample obtained from a subject which comprises the steps of:
(i) centrifuging the serum or plasma sample to obtain a supernatant liquid; and
(ii) analysing the supernatant liquid for a cell free nucleoprotein chromatin fragment.

2. The method according to claim 1, wherein the cell free nucleoprotein chromatin fragment comprises a nucleosome.

3. The method according to claim 2, wherein the nucleosome comprises an epigenetic structure selected from the group consisting of a histone post-translational modification, a histone variant, a histone isoform, a DNA modification to the nucleosome and a protein adducted to the nucleosome.

4. The method according to claim 2, wherein the nucleosome comprises a histone isoform selected from H3.1 or a histone post-translation modification selected from H3R8-Cit.

5. The method according to claim 1, wherein the serum or plasma sample is centrifuged at a relative centrifugal force of more than 5000×g.

6. The method according to claim 1, wherein the serum or plasma sample is centrifuged for between 1 and 10 minutes.

7. The method according to claim 1, wherein the serum or plasma sample is centrifuged for about 2 minutes at a relative centrifugal force of between 5000 and 15000×g.

8. The method according to a m claim 1, further comprising centrifuging a whole blood sample obtained from the subject to separate the blood cells from the serum or plasma and transferring the serum or plasma to a separate container prior to further centrifugation.

9. The method according to claim 8, wherein the whole blood sample is centrifuged at a relative centrifugal force of 1500-3000×g.

10. The method according to claim 8, wherein the whole blood sample is centrifuged between 5 and 15 minutes.

11. The method according to claim 1, wherein the serum or plasma sample is frozen and subsequently thawed prior to step (ii).

12. A method for assessing or detecting a disease state of a subject which comprises the steps of:
(i) centrifuging a serum or plasma sample obtained from the subject to obtain a supernatant liquid;
(ii) analysing the supernatant liquid for a presence or an amount of nucleoprotein chromatin fragment; and
(iii) using the presence or the amount of nucleoprotein chromatin fragment as an indicator of the disease state of the subject.

13. The method according to claim 12, wherein the disease state is selected from the group consisting of a cancer, an infection, an autoimmune disease and an inflammatory disease.

14. The method according to claim 3, wherein the nucleosome comprises a histone isoform selected from H3.1 or a histone post-translation modification selected from H3R8-Cit.

15. The method according to claim 14, wherein the serum or plasma sample is centrifuged at a relative centrifugal force of more than 5000×g.

16. The method according to claim 14, wherein the serum or plasma sample is centrifuged for between 1 and 10 minutes.

17. The method according to claim 14, wherein the serum or plasma sample is centrifuged for about 2 minutes at a relative centrifugal force of between 5000 and 15000×g.

18. The method according to claim 2, further comprising centrifuging a whole blood sample obtained from the subject to separate the blood cells from the serum or plasma and transferring the serum or plasma to a separate container prior to further centrifugation.

19. The method according to claim 18, wherein the serum or plasma sample is frozen and subsequently thawed prior to step (ii).

20. The method according to claim 3, further comprising centrifuging a whole blood sample obtained from the subject to separate the blood cells from the serum or plasma and transferring the serum or plasma to a separate container prior to further centrifugation.
